(19) United States
(12) Patent Application Publication
(10) Pub. No.: US 2005/0246105 A1
(43) Pub. Date: Nov. 3, 2005

(54) Interpolated image response

(76) Inventors: Vance Faber, Carnation, WA (US); John W. Elling, Santa Fe, NM (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 11/044,395

(22) Filed: Jan. 27, 2005

Related U.S. Application Data
(60) Provisional application No. 60/539,322, filed on Jan. 28, 2004.

(51) Int. Cl. G06F 19/00; G01N 33/48; G01N 33/50

(52) U.S. Cl. 702/19; 702/20

(57) ABSTRACT

Systems and methods are provided for characterizing a multidimensional distribution of responses from the objects in a populations subject to a perturbation. The methods enable the creation of a “degree of response” scale interpolated from non-perturbed and perturbed reference populations. The methods enables, using the interpolated degree of response scale, the quantitation of a degree of response of a test compound subject to a given level of perturbation, and enables the generation of a dose-response curve for a test compound. The methods are useful in a wide range of applications, such cellular analysis and high-content screening of compounds, as carried out in pharmaceutical research.

### CROSS-REFERENCE TO RELATED APPLICATIONS

The present application claims priority to U.S. application Ser. No. 60/539,322, filed Jan. 12, 2004, which is incorporated herein by reference in its entirety.

### STATEMENT REGARDING FEDERALLY SPONSORED RESEARCH OR DEVELOPMENT

This invention was made in part with Government support (DHHS Grant No. 1 R44 NS45384-01). The Government may have certain rights in the invention.

### BACKGROUND OF THE INVENTION

1. Field of the Invention

The present invention relates in general to systems and methods for characterizing and comparing images. More particularly, the invention relates to systems and methods for comparing and analyzing images of biological substances, in particular, cells.

2. Description of Related Art

Assays for monitoring biological effects due to a perturbation are commonly used in drug discovery, diagnostics, and predictive medicine to determine efficacy, toxicity, or other biology responses. Due to the complex nature of a biological response, typically an assay is designed to provide quantitative measures of one or more specific changes known to be associated with either the tested perturbation or analogous perturbation. For example, where the perturbation is caused by exposure to a drug, it is typical to subject the sample to a concentration range of the compound and monitor the extent of effect on the sample, and the parameters measured are selected to be particular biological features that are, a priori, expected to provide a biologically meaningful measure of response, such the expression and/or localization of a known protein. The resulting parameter values are plotted graphically and used to estimate effective dosages of the compound. Because the assay is designed to monitor only specific expected effects, the information obtainable from the data regarding the full-scope of the biological effect of the compound is inherently limited. Examples of such assays, in particular, assays designed to measure protein translocation, are described in Ding et al., 1998, Journal of Biological Chemistry 273(44): 28897-28905; and Giulano et al., 1997, Journal of Biomolecular Screening 2(4):249-259.

Pattern recognition is a powerful tool for comparing images of biology samples and identifying a similarity or difference due to perturbation. This approach removes the limitations of knowing and developing specific assays that measure one or more parameters of known biological significance, and instead, monitors a plurality of cellular attributes, conditions, and changes with a minimal a priori knowledge about the effects. A major challenge associated with this approach is the interpretation and representation of the data derived from pattern recognition-based analysis.

### BRIEF SUMMARY OF THE INVENTION

The present invention provides systems and methods for characterizing and comparing responses of populations of objects subject to a perturbation, wherein a response refers to a multidimensional distribution of object features.

The methods of the present invention enable, based on the multidimensional distribution of object features that characterize each of a non-perturbed and a perturbed reference population, the creation of a “degree of response” scale that provides multidimensional statistical descriptions for a series of populations at intermediate degrees of response. One aspect of the invention is the definition of a “degree of response” for responses that are multidimensional statistical descriptions of objects in a population; a second aspect of the invention is the generation of a interpolated degree of response scale.

The degree of response scale enables the determination of a quantitative degree of response of an empirically determined response of a test population to a given level of perturbation. Further, the degree of response scale enables the generation of a dose-response curve for a test compound, wherein the response of a test population is determined at multiple levels of perturbation. Another aspect of the invention is method(s) of determining the degree of response of a test population from an interpolated degree of response scale, and an additional aspect of the invention is the generation of a dose-response curve for a test compound.

The present invention is particularly applicable to, but not limited to, biological applications. In preferred embodiments, the present invention provides systems and methods for analyzing cellular samples that have been exposed to a perturbation, such as a drug, toxin, signaling protein, or other bioactive compound, wherein statistical pattern recognition is used to analyze images of the samples. The present invention enables the determination of a degree of response of a cellular sample subject to a known perturbation, and enables the determination of a dose-response curve that characterizes the degree of response of cellular samples subject to different levels of perturbation.

In a preferred embodiment, a degree of response scale is determined from reference samples representing the sample response at the endpoints of the range of perturbations of interest. Typically, a perturbation in a cellular assay refers to a bioactive compound that is applied to the sample, and the range of perturbations refers to the range of concentration at which the compound is applied. The reference samples, each containing at least one, but preferably many cells, are assayed under conditions that define the endpoints of the range of perturbations of interest. Typically, one sample will represent the unperturbed state (i.e., no compound applied), and the other sample will represent a “maximally” perturbed state, although the methods are equally applicable to subranges of the possible perturbation levels. A multidimensional statistical description of the cells within each sample, referred to as a “fingerprint” of the sample, is obtained from, for example, a pattern-recognition analysis of one or more images of each sample. Thus, these reference populations provide fingerprints characterizing the state of the cells within the population (i.e., the response of the population) at the low and high endpoints of the range of perturbations.

Given the two reference population fingerprints, one representing the response at the lowest perturbation and one representing the response at the highest perturbation, a degree of response scale is generated that represents estimates of the response (i.e., fingerprints) of populations subject to intermediate levels of response. To define the endpoints along the degree of response scale, the lowest response is set to equal the response (i.e., fingerprint) at the lowest perturbation, and the highest response is set to equal the response (i.e., fingerprint) at the highest perturbation. For convenience, the range of the degree of response scale arbitrarily is set to be the interval from zero to one (equivalently, from 0% to 100%) by setting the lowest response to be zero and the highest response to be one.

The degree of response scale is generated from the endpoint responses using a mathematical model of the change in the response. Example classes of models are provided for describing intermediate responses in cellular assays, based on reasonable assumptions about the biology of cellular responses.

The present invention provides methods of using the degree of response scale to quantitate an empirically determined response (fingerprint) of a test population to a known perturbation. The empirically determined test response is compared to the interpolated responses to find the most similar interpolant, and the degree of response of the test population to the perturbation is assigned a degree of response corresponding to the most similar interpolant. Methods are provided for calculating the most similar interpolant. In some embodiments, a set of interpolants are generated from the model, and the test fingerprint is compared to the generated interpolants. In preferred embodiments, the most similar interpolant is identified analytically from the interpolant model.

The present invention further provides methods of calculating a dose-response curve, which describes the relationship between the response of a population and the level of perturbation, e.g., the concentration level of compound administered to the sample. Quantitating the responses of a series of test populations, each exposed to a different concentration of the compound, using the degree of response scale, provides a series of points from a dose-response curve. This series of points can be plotted to provide a standard 2-dimensional dose-response plot for the test compound. The empirically determined points can be fitted to a curve to obtain a dose-response curve. The dose-response curve, defined for multi-dimensional responses, can be used in a manner that is analogous to a standard, single-parameter, dose-response curve.

The methods of quantitating the response of a test population relative to a interpolated degree of response scale further provides a method of assessing the response of a test population with respect to multiple degree of response scales, each generated from reference populations subject to different kinds of perturbations. For example, the methods allow comparing the effect of a new drug candidate with respect to the effects of multiple known drugs. Interpolated degree of response scales can be generated for each of the known drugs, and the response of a test population subject to the candidate drug can be compared to each of the degree of response scales. The degree of response obtained from each interpolated scale provides a measure of the similarity of the effect of drug candidate relative to each known drugs.

In measuring the response of a test population relative to multiple degree of response scales, the distance from the test population to the most similar (closest) interpolant in a scale provides a measure of how well the response of the test population is characterized by that scale. Conceptually, a test response consists of a component response along a degree of response scale and a component response away from the scale. A scale well characterizes a test response if the portion of the response along the scale is maximized and the portion of the response away from scale is minimized. Thus, for example, comparing the response induced by drug candidate to the scales obtained from a number of known drugs, the drug candidate can be considered most similar to the drug corresponding to the scale that best characterizes the response of the test sample.

The present invention also provides systems for carrying out the methods of the invention. Such systems typically provide an instrument for acquiring multidimensional measurements of the objects in a population of objects, and a computer containing instructions on a machine-readable medium for carrying out the methods of the invention on the acquired data. In a preferred embodiment, the system of the invention comprises elements that allow for the automated analysis of samples, and comprises the image acquisition module, such as an automated digital microscope, and a computing module that enables the analysis of the image data obtained using the methods of the present invention.

The systems and methods described herein are broadly applicable to drug discovery, diagnostics, pathology and predictive medicine, as well as non-biological fields wherein blending pattern recognition-derived image data can provide a predictive estimations of intermediate values. Such fields include, but are not limited to, facial recognition, fingerprint analysis, retinal scans, and handwriting.

### DETAILED DESCRIPTION OF THE INVENTION

The following definitions are provided for clarity. Unless otherwise indicated, all terms are used as is common in the art. All reference cited herein, both supra and infra, are incorporated herein by reference.

As used herein, “system” and “instrument” are intended to encompass both the hardware (e.g., mechanical and electronic) and associated software (e.g., computer programs) components.

An “object” is used herein to refer to the individual elements in a sample from which feature measurements are made. The definition of an object is assay-dependent and is not a critical aspect of the invention. Typically, the nature and intent of an assay, along with the measurement capabilities of the instrumentation, will determine what sample component is selected as an object. For example, in a cellular assay in which measurements of individual cells are obtained, an object is defined as a single cell.

A “sample” or “population” is used herein to refer to a collection of at least one, but preferably many objects.

The terms “descriptors”, “features”, “primitives”, and “statistics” are used herein to refer to individual parameters measured or calculated from objects. An object feature can be a measurement taken directly from the object, such as a dimension, color, or luminosity, or can be a function or statistic of the measurements, such as the area, moments (e.g., centroid, variance, skewness, kurtosis), or texture; measured either from the object as a whole, or from a subcomponent of the object. The choice of a suitable set of descriptors depends on the application, and one of skill in the art will be able to select a suitable set following the teaching herein. The set of features used to measure objects is represented herein as forming a multidimensional feature space, and measurements of features from one object resent a point in the multidimensional feature space.

I. Fingerprints

The term “fingerprint”, as used herein, broadly refers to a multidimensional description of an object or a sample containing a plurality of objects, or, equivalently, an image of an object or sample, in terms of a set of descriptors or features.

A fingerprint of an object, such as a cell, also referred to herein as a feature vector, refers herein to a vector of descriptor (feature) values that characterize the object. Conceptually, a fingerprint of an object can be represented as point in the multidimensional feature space.

A fingerprint of a population containing a plurality of objects refers to the set of object fingerprints, or to a representation of the distribution of object fingerprints. Conceptually, a fingerprint of a population can be represented as a distribution in the multidimensional feature space. The set of object fingerprints of a sample can be represented conveniently as a two-dimensional array of descriptor values, xᵢⱼ, wherein xᵢⱼ is the value of the jth descriptor measured from the ith object, i.e., an array in which each row is a feature vector for one of the objects. The distributions of each of the features can be calculated from the array of feature vectors. Alternatively, the fingerprint of a population can be represented as a set of, or vector of, the individual feature distributions. For example, a fingerprint can be represented by histograms of the values observed for each feature, or by a distribution function, typically obtained by fitting the observed data to a distribution. In some embodiments, the representation of the fingerprint as an array of feature vectors (i.e., by a set of points in the feature space) is preferred as it facilitates the use of resampling methods to estimate population fingerprint distributions.

The term “Cytoprint™” (a trademark of Atto Bioscience, Rockville, Md.) refers to a fingerprint of a sample of cells. Although the present invention is particularly applicable to cellular assays, and the invention is described herein in detail as applied to cellular assays, it will be clear to one of skill that the present invention is not limited to cellular assays, but is applicable to the analysis of features of populations of objects in general.

Methods of measuring a fingerprint of a sample (or an image of a sample) are described in copending U.S. application Ser. No. 10/116,640, published as U.S. patent application publication No. U.S. 2002/0159625, incorporated herein by reference. Both the selection of features that constitute a fingerprint in a particular application and the method of generating the fingerprint applicable to that application are not critical aspects of the present invention. Preferred methods are described herein as examples, and it will be clear to one of skill that the present invention is not limited to the exemplified methods and applications.

Typically, a fingerprint of a sample, e.g. a sample of cells, is obtained using the following steps:
1. obtaining a digital image of the sample;
2. identifying objects within the sample (referred to as “image segmentation”); and
3. determining the values of a feature vector for each of a plurality of objects contained in the sample;
4. storing the feature values in a machine readable form.
   The resulting fingerprint of the sample is the collection of feature vectors. Optionally, a histogram or distribution of feature values for each of the plurality of features can be derived from the fingerprint.

An image of a sample may be obtained using any suitable means. In a preferred embodiment, an image of a sample of cells is obtained using a digital-imaging microscope, preferably a confocal microscope. Suitable microscopes are available commercially from a number of vendors, such as, for example, BD Biosciences, Bioimaging systems (Rockville, Md.), Amersham Biosciences (now part of GE Healthcare; Piscataway, N.J.), Carl Zeiss Inc. (Thomwood, N.Y.), Olyumpus (Melville, N.Y.), Molecular Devices (Sunnyvale, Calif.), Cellomics (Pittsburgh, Pa.), Evotech Technologies GmbH (Hamburg, Germany), and Beckman Coulter (Fullerton, Calif.).

Methods of identifying regions within an image (“image segmentation”) corresponding to either objects within a sample or subregions of objects are well known. For example, “Digital Image Processing” by Rafael C. Gonzalez and Paul Wintz, second edition, 1987, is a textbook that describes various image processing techniques, including segmentation, and is incorporated by reference herein in its entirety. One of skill will be able to select methods suitable to a particular application from the extensive descriptions in the literature.

Methods for determining the values of a feature vector for each of a plurality of objects contained in the sample depend on the features selected for the particular application. Typically, the feature values are obtained by direct measurement of the segmented image, followed by calculation of the appropriate statistic or function. For example, the area of an object in a digital image is obtained typically by counting the number of picture elements (pixels) contained within the image of the object, optionally relating this to the physical area represented by a pixel.

In some embodiments, fingerprints may be defined based on a subset of the features actually measured. This can be desirable if it is known a priori that particular features, although measured, are not of interest in the particular application, or if data obtained from particular features from some or all of the populations assayed are anomalous. For example, in a cellular assay, the emission of a fluorescent dye used solely to facilitate identification of a subcellular component, such as a nucleic acid stain used to locate the nuclear region, may not provide, in some applications, a meaningful measurement of cellular response.

II. Perturbations

The term “perturbation” is used here to refer to any measurable parameter that has the potential to cause an observable change in a sample or population of objects. As used herein, the perturbation refers to the treatment of the sample, not to the response of the sample. The nature of the perturbation is not a critical aspect of the invention and the present methods are broadly applicable. Perturbations can comprise a breadth of conditions that influence the sample and include, but are not limited to, any one or more of the forces selected from the group consisting of chemical, biological, mechanical, thermal, electromagnetic, gravitational, nuclear, and temporal.

The level of perturbation, as used herein, refers to some scalar measure of the amount of perturbation applied to the sample. An appropriate measure of the level of perturbation depends on the nature of the perturbation. For example, in a biological assay, the perturbation typically is a bioactive compound, such as a drug, hormone, toxin, or agonist, and the concentration of the compound is a suitable measure of level of perturbation applied to the sample. Alternatively, the perturbation can be a single concentration applied to samples for various lengths of time, and an appropriate measure of the level of perturbation is the application time. In another embodiment, the perturbation can be a discrete event followed by a period of time to allow the objects to respond, and the measure of the level of perturbation is the time following the perturbation event.

Where the fingerprint of a population is to be determined at multiple levels of perturbation, it should be clear that this is carried out using replicate samples of the population, each exposed to one of the levels of perturbation.

III. Responses

As used herein, the “response” of an object or population subject to a given perturbation refers to the state of the perturbed object or population. The response is measured as a fingerprint of the perturbed object or population.

The response need not be measured with respect to a reference, unperturbed sample, as the response refers to the state of the perturbed sample, rather than the change in the state of the sample due to perturbation. However, various measures of distance between fingerprints, as described herein, can be applied to the fingerprints from differently perturbed samples to provide a measure of distance between a reference and a perturbed sample.

IV. Dose-Response

The term “dose-response curve” is used herein in general to describe the relationship between the degree of response of a population and the level or perturbation applied to the population. In the present context, a response refers to the multidimensional statistical characterization of objects in the population (a multidimensional distribution in feature space), and one aspect of the invention is the definition of, and calculation of, a “degree of response” in this context.

The term “EC50” refers to the perturbation level that provokes a response halfway between baseline response and maximum response.

V. Degree of Response Scale

In one aspect, the present invention provides methods for generating a “degree of response” scale (also referred to herein as simply a response scale) that represents the fingerprints of populations at intermediate degrees of response. The degree of response scale is interpolated from the response endpoints, which are the responses of the minimally and maximally perturbed populations, respectively. The intermediate-response fingerprints are referred to herein as interpolants or interpolated fingerprints. The degree of response scale refers to the set of interpolants, along with the empirically determined endpoints, indexed by the corresponding degree of response.

It should be noted that the degree of response scale defines a curve of unit length in the space of distributions in feature space connecting the reference fingerprints, wherein the distance along the curve from an unperturbed reference fingerprint is a measure of the degree of response.

The endpoints of the degree of response scale are defined from the fingerprints of the reference populations; the lowest response is defined as the response at the lowest level of perturbation, and the highest response is defined as the response at the highest level of perturbation. Typically, the reference populations will represent the unperturbed state and a “maximally” perturbed state, although the methods are equally applicable to subranges of the possible perturbation levels. Similarly, the lowest response typically is assumed to represent a “zero” response and the highest response typically is assumed to represent a maximum response, although the methods are equally applicable to subranges of responses. For convenience, the range of the degree of response scale typically is set arbitrarily to be the interval from 0 to 1 (equivalently, from a 0% to 100% response), although other intervals, such as the interval from 0 to −1 may be more convenient in some cases (e.g., for an antagonistic perturbation). In embodiments wherein it is known that the maximal observed response does not represent a truly maximal response (e.g., wherein the highest level of perturbation applied results in a change in only a portion of the objects in a sample), it may be desirable rescale the range of the response index accordingly.

Typically, population responses can vary in a continuous manner, and the degree of response is a continuous variable ranging from 0 (no response) to 1 (full response) or, equivalently, from 0% to 100% response. In this case, the degree of response scale comprises an infinite set of interpolants, each indexed by the degree of response. In some embodiments, population responses will be limited to a finite number of possible discrete states, and the degree of response scale will comprise a finite set of interpolants.

In some embodiments, a degree of response scale is approximated by a set of responses along the scale. Such approximations can reduce the computation and storage required in some embodiments of the invention, for example, in which interpolants are generated and stored using resampling methods, and can also be desired in general due to inherent limits on the level of precision that is meaningful for parameters calculated from experimental results. The number of interpolants generated will be determined by the desired step size in the degree of response, and will depend on the application. For example, in some cases, it may be sufficient to generate or consider interpolants corresponding to integer changes in the percent response (i.e., 0%, 1%, 2%, . . . 100% response). Alternatively, for embodiments of the invention in which exact results are obtained, the results may rounded off to an appropriate precisions.

VI. Interpolant Models

For each degree of response, the corresponding interpolant is obtained from a model of the change in a feature vector distribution with increasing response. An appropriate model depends on the application and, in particular, the nature of the objects and perturbation applied. One of skill in the art will be able to select an appropriate model for a particular application following the teaching herein. Modeling a process, such as the response of an object to a perturbation, typically involves an approximation or simplification of the actual process, whose mechanism may be unknown or incompletely understood. Models may be based on a known or assumed underlying mechanism, or may be a purely phenomenological model in which an outcome is predicted from an input using, for example, an empirically determined relationship. Models will be useful in the methods of the present invention according to their predictive value, whether or not a model reflects, or is based on, an underlying mechanism.

A preferred application of the present invention is in the analysis of samples of cells subjected to a perturbation such as a bioactive compound. A preferred class of models of intermediate fingerprints in cellular assays is obtained by assuming an underlying model of cellular response in which cells have only two states, unperturbed and fully perturbed (e.g., unactivated and activated), and that the probability of a cell changing state (e.g., becoming activated) is a function of the concentration of the perturbing compound. This model of the underlying biology may be applicable to a wide range of cellular assays treated with pharmaceutical compounds or toxins. For example, a compound that induces apoptosis may function by triggering a cascade of intracellular events that results in a complete state change of the cell (i.e., becoming apoptotic), wherein the fraction of cells triggered depends on the concentration. Similar behavior may be expected from a wide range of compounds that trigger intracellular signaling cascades, or, more generally, that effect a polarized cellular response.

A model of intermediate-response sample states is obtained from the assumptions on the underlying biology as follows. It is assumed that a small fraction, or none, of the cells in the no-response reference sample are in the perturbed state, and that a larger fraction, or all, of the cells in the maximum-response reference sample are in the perturbed state. An intermediate-response population corresponds to a population containing an intermediate number of cells in the perturbed state, and can be represented as a mixture of the reference populations. Let the probability density functions of the no-response (0 on the degree of response scale) and full-response (1 on the degree of response scale) reference populations be designated f₀ and f₁, respectively. Let f_{α} be the probability density function of a population having an intermediate response equal to α, where α is a function of the level of perturbation taking values between 0 and 1. Then, a class of models, indexed by the function α, describing the density function of a population having an intermediate response is defined as
f_{α}(x)=αf₁(x)+(1−α)f₀(x).
The fingerprint of a population having an intermediate response equal to α is referred to herein as the α-interpolant.

The value of α is a measure of the degree of response. Conceptually, in terms of the degree of response curve in feature space, α measures the displacement along the curve from the no-response to the full-response reference populations. No assumptions are made about the function α other than it is a function of the level of perturbation taking values between 0 and 1. In fact, α, as a function of the concentration, represents a dose-response curve. The present invention provides methods of determining the functional form of α by comparing empirically determined responses of samples subject to known concentrations to the modeled degree of response scale.

An alternative class of models of intermediate fingerprints in cellular assays is obtained by assuming an underlying model of cellular response in which there is a continuum of cellular states between unperturbed and fully perturbed (e.g., unactivated and activated), and that all cells in the intermediate population are in the same intermediate state. In this class of models, the state of the cells is a function of the concentration of the perturbing compound. This assumption of continuous cellular responses in the underlying biology may be applicable to some cellular processes, such as, for example, cell size in response to a growth factor. The use of this class of models is described in the examples, below.

The preferred class of models based on an underlying two-state model of cellular response has been found to be useful in a number of cellular assays. It is expected that in some particular cases, depending on the cellular features measured, the class of models based on an underlying continuously-varying cellular response, or another class of models based on different assumptions of the underlying biological processes, will provide more useable results. Because of the complex, varied, and typically poorly understood nature of cellular processes, it is expected that the suitability of a class of model will be application dependent. Furthermore, it will be understood that for any model of a biological process, the accuracy of representation preferably should be determined by comparison with empirically determined results.

VII. Use of a Degree of Response Scale to Score a Test Sample

The present invention provides methods of using the degree of response scale to quantitate an empirically determined response (fingerprint) of a test population to a known perturbation. As described in detail, below, the empirically determined response is compared to the interpolated responses to find the “most similar” interpolant, and the degree of response corresponding to the most similar interpolant is reported as the degree of response of the test population. Thus, the degree of response scale provides a quantitative degree-of-response score for a test population fingerprint based on the two reference population fingerprints.

Because the fingerprints are distributions in a multidimensional feature space, and a test compound fingerprint can deviate from a reference fingerprint in any or all of these dimensions, it is highly unlikely that a fingerprint of a test compound will coincide with one of the interpolates. For this reason; the similarity of a test population fingerprint to an interpolant is measured using a distance metric defined for distributions in the feature space. Given a suitable metric of the distance between a fingerprint and an interpolant, the most similar interpolant in the degree of response scale is obtained by determining the interpolant that minimizes the distance between the test fingerprint and the interpolants in the degree of response scale.

A. Distance Metrics for Distributions

A number of metrics are known in the literature that are suitable for measuring the distance between multidimensional distributions of feature vectors. For example, distance metrics that have been proposed in computer vision applications for measuring the distance between images characterized as distributions in a multidimensional feature space, which may be useful in the present invention, include heuristic measures, such as the Minkowski-Form distance, Histogram Intersection, and Weighted-Mean-Variance; nonparametric test statistics, such as the Kolmogorov-Smirnov distance, Cramer/von Mises (squared Euclidean distance), and the χ² statistic; information-theory divergences, such as the Kullback-Leibler divergence and Jeffrey-divergence; and ground distance measures, such as the Quadratic Form and the Earth Movers Distance (see, for example, Rubner et al., 2001, Computer Vision and Image Understanding 84:25-43, and Rubner et al., 2000, International Journal of Computer Vision 40(2): 99-121, both incorporated herein by reference).

In a preferred embodiment, the distance between two fingerprints (or between a fingerprint and an interpolant) is based on the Kolmogorov-Smirnov statistic. The Kolmogorov-Smirnov (KS) distance between two one-dimensional distributions (or histograms) is the maximal discrepancy between the cumulative distribution functions (or histograms). Thus, the KS distance, D, between two cumulative distribution functions, F₁ and F₂, is defined by

$$D = {max}_{y} \vert {F}_{1} ( y ) - {F}_{2} ( y ) \vert .$$

Equivalently the KS distance between two continuous probability density functions, f₁ and f₂, is defined by

$$D = {max}_{y} \vert {\int }_{- ∞}^{y} [ {f}_{1} ( x ) - {f}_{2} ( x ) ] ⅆ x \vert .$$

The Kolmogorov-Smirnov distance is a measure for unbinned distributions and, thus, avoids the problems of data binning encountered using distance metrics that compare histograms on a bin-by-bin basis.

The Kolmogorov-Smirnov statistic is defined only for one dimension. To measure the distance between multidimensional fingerprints of two populations, the KS distance is used to measure the distance between the two populations for each feature separately, and the KS distance between the fingerprints is defined as the maximum of the KS distances from the features. Thus, the KS distance, as defined herein for fingerprints, is the maximum of the individual feature KS distances.

In some embodiments, the fingerprint is stored as a set of object feature vectors, which represents a set of points in the feature space, and the cumulative feature distributions or histograms are calculated from the data at the time the distance is measured. Typically, a cumulative histogram of feature values is obtained using the data contained in the entire fingerprint. However, to reduce the computation required for estimating distance between particularly large populations, the KS distance can be estimated using a random sampling of feature value data from the fingerprint.

B. Scoring a Test Compound

To quantitate an empirically determined response of a test population subject to a known level of perturbation, the interpolant that minimizes the distance between the test fingerprint and the interpolants in the degree of response scale is determined, and the degree of response of the closest interpolant is reported as the degree of response of the test fingerprint along the degree of response scale. The minimum distance interpolant can be determined in a number of ways, as summarized below and described in more detail in the examples.

In one embodiment, a suitable number of interpolants are generated from the model and stored in a system readable memory. To find the minimum distance interpolant, the distance from the test fingerprint to each of the interpolants is measured using the selected distance metric, preferably the KS distance.

In preferred embodiments, the interpolants are not actually generated and stored, but rather the closest interpolant is identified algorithmically using the underlying model of the interpolants and the endpoint fingerprints. Algorithms for determining the closest interpolant under two interpolant models described above are set forth in the examples.

In a preferred embodiment, multiple replicates of a test sample are assayed, the degree of response measured for each replicate separately, and the mean response and standard error of the response are reported.

VIII. Dose-Response Curves

A dose-response curve is estimated empirically by quantitating the responses of a series of test populations, each exposed to a different concentration of the compound, using the degree of response scale. This series of points from the dose-response curve can be plotted to provide a standard 2-dimensional dose-response plot for the test compound, and the empirically determined points can be fitted to a curve to obtain a dose-response curve. The dose-response curve, defined for multi-dimensional responses, can be used in a manner that is analogous to a standard, single-parameter, dose-response curves. In particular, an EC50, which represents the perturbation level that provokes a response halfway between baseline response and maximum response, can be obtained from the dose-response curve using standard methods.

### EXAMPLES

The following examples are put forth so as to provide one of ordinary skill in the art with a complete disclosure and description of how to make and use the present invention, and are not intended to limit the scope of what the inventors regard as their invention. The examples describe applications of the present invention to cellular analyses. However, the particular application, methods, instruments and systems described in the following examples are exemplary, and should not be considered limiting.

The following examples include mathematical descriptions of embodiments of the present invention. It will be understood that implementation of the methods typically will on a programmable computer. The programming of mathematical algorithms is well known in the art, and tools for writing such programs, such a programming languages and libraries of mathematical functions, are commercially available from a large number of sources. One of skill in the art will be able to translate routinely the mathematical descriptions contained herein into an appropriate set of instructions using any of a number of commonly used programming languages.

### Example 1

### Generating a Degree of Response Scale by Resampling

This example describes a method for scoring test sample using a degree of response scale generated from the low-response and high-response reference samples by resampling.

The model of intermediate fingerprints used herein is based on an underlying two-state model of cellular response. More specifically, given the distributions of the no-response (0 on the degree of response scale) and full-response (1 on the degree of response scale), designated f₀ and f₁, respectively, and the distribution of a population exhibiting an intermediate response equal to α, designated f_{α}, then the distribution of the intermediate-response population is f_{α}(x)=αf₁(x)+(1−α)f₀(x).

The distribution of the population having intermediate-response α is estimated by creating a virtual population comprising a portion at of feature vectors chosen at random with replacement from the High population fingerprint, and a portion (1−α) of feature vectors chosen at random with replacement from the Low population fingerprint. Preferably, the total size of the resampled intermediate population (i.e., the total number of feature vectors) is chosen to be the size of the reference populations. If the size of the reference populations are not equal, a subset of the feature vectors from the larger reference population can be selected to provide equal size reference populations. Interpolant distributions are generated for no more than N discrete equally-spaced values of α, where N is the sample size of the resampled population.

The nearest interpolant to a test fingerprint can be determined by a brute force method in which the distance to each of the interpolants is measured and the minimum selected. Preferably, the nearest interpolant is determined using a more efficient algorithm, such as a standard bisection algorithm.

In one embodiment, the interpolant populations thus generated are stored for use in comparing with one or more test sample fingerprints. In this case, although the storage requirements may be high, the resampling process needs to be carried out only once for each level of α. Alternatively, the interpolant populations can be generated and stored in temporary memory each time a distance to a test population is measured. This may be desirable to minimize memory requirements, particularly when used with a bisection algorithm in which only a subset of the interpolant typically need to be compared with the test fingerprint to find the nearest.

### Example 2

### Scoring Directly from Low- and High-Response Histograms

This example describes algorithms for scoring test images directly from the low-response and high-response reference samples.

A model of intermediate fingerprints used herein is based on an underlying two-state model of cellular response. More specifically, given the distributions of the no-response (0 on the degree of response scale) and full-response (1 on the degree of response scale), designated f₀ and f₁, respectively, and the distribution of a population exhibiting an intermediate response equal to α, designated f_{α}, then the distribution of the intermediate-response population is f_{α}(x)=αf₁(x)+(1−α)f₀(x).

The algorithm herein will be described in terms of feature histograms from the sample fingerprints, which represent discrete-valued approximations of the underlying distributions. For convenience, it is assumed that the fingerprint of a sample, which is the set of object fingerprints of the sample, is be represented as a two-dimensional array of descriptor values, i.e., an array in which each row is a feature vector for one of the objects. Thus, for example, a fingerprint is denoted as a set of data, {xᵢⱼ} wherein xᵢⱼ is the value of the jth descriptor measured from the ith object. Instead of mixing the samples from the no-response and full-response reference populations (referred to as the Low and High distributions), as in the methods of Example 1, the present algorithm mixes the cumulative distributions (histograms).

Let {xᵢⱼ}, {yᵢⱼ}, {zᵢⱼ} denote the data from the two references images and the test image, where {xᵢⱼ} represents the data from the Low, {yᵢⱼ} represents the data from the High, and {zᵢⱼ} represents the data from the Test image.

Fix j and let sⱼ be a member of S={xᵢⱼ}∪{yᵢⱼ}∪{zᵢⱼ}, that is, sⱼ is one of the possible values of the jth statistic. Assuming S is sorted, define cumulative histograms for each feature as
H(sⱼ)=|{zᵢⱼ:zᵢⱼ<sⱼ}ᵢ₋₁^{N}|/N,
F(sⱼ)=|{yᵢⱼ:yᵢⱼ<sⱼ}ᵢ₌₁^{M}/M
and
G(sⱼ)=|{xᵢⱼ:xᵢⱼ<sⱼ}ᵢ₌₁^{L}|/L,
where | . . . | denotes the cardinality of the set, and N, M, and L are the total number of cells in the respective samples.

The KS distance between the Test image distribution, H, and the α interpolant distribution is defined to be

$$D ( α ) = {max}_{j} {max}_{{s}_{j}} \vert u ( {s}_{j} ) - α v ( {s}_{j} ) \vert ,$$

where
u(sⱼ)=H(sⱼ)−G(sⱼ)
and
v(sⱼ)=F(sⱼ)−G(sⱼ).
The desired distance from test image to the closest interpolant is,

$$D = {min}_{α} D ( α ) .$$

Methods for Finding the Minimum α and α-Interpolant

In the following methods, the distance, D(α), between any the test distribution and an α-interpolant distribution is calculated from the reference and test fingerprints using the KS distance, as described above.

a. Bisection

In one embodiment, the location and value for the minimum can be determined using a standard bisection algorithm.

Alternatively, the degree of response scale is approximated by a finite subset of α-interpolants by assuming a takes on only a finite set of discrete intermediate values, and the distance, D(α), is evaluated only at these discrete intervals. This approximation can significantly reduce the computation required to find the minimum distance using a bisection algorithm.

b. Linear Programming

In another embodiment, the minimum distance is obtained using linear programming. The problem of finding the closest interpolant outlined above reduces to a general problem of this type:

$$D = {min}_{0 \leq α \leq 1} {max}_{k} \vert {u}_{k} - α {v}_{k} \vert$$

where the pairs (uₖ,vₖ) come from a finite set.
This problem can be stated and solved as a problem in Linear Programming as follows. Note that if the solution D occurs at the value α=α₀, then
D≧|uₖ−α₀vₖ| for every k.
Thus (D,α₀) is the solution to the LP problem:
min Y(y,α) with Y(y,α)=y subject to
   y−uₖ+vₖα≧0
   y+uₖ−vₖα≧0
   α≦1
   α≧0.
   In particular, if we include the pair (−uₖ,−vₖ) in the set whenever the pair (uₖ,vₖ) is in the set, then we can write the problem as:
min Y(y,α) with Y(y,α)=y subject to
y−uₖ+vₖα≧0
α≦1
α≧0.
We can use a type of simplex method, described below, to solve this problem.
Algorithm. Consider the two-dimensional constraint region with horizontal axis a and vertical axis y. This convex region is bounded by the vertical lines at α=0 and α=1 and lies above all the lines y=−vₖα+uₖ. We will start on the constraint boundary at α=0 and y=max uₖ. Let us assume that the constraint which provides this maximum is called (u₀, v₀). If there is more than one constraint passing through α=0 and y=u₀, we choose the one with the smallest v and hence the largest slope. We follow the constraint (u₀,v₀) until it meets another (u,v) at α=α₀. Then we remove all the constraints (uⱼ,vⱼ) with uⱼ>u because they must meet the constraint (u,v) before α=α₀. Let us assume that this new constraint is called (u₁,v₁). We iterate this procedure with the remaining constraints until either we hit the boundary at α=1 or Y(y,α) starts to increase.
More specifically, we give a method for determining which constraint is the first to meet the constraint (u₀,v₀). To analyze the situation, we solve for the intersection of the constraint (u₁,v₁) and the constraint (u₀,v₀). If these two constraints meet at α=α₀>0 then

$${α}_{0} = {min}_{( u , v )} \frac{{u}_{0} - u}{{v}_{0} - v}$$

and (u₁,v₁) is the (u,v) pair which achieves this minimum.
In particular, since u₀>u₁, we must have v₀>v₁. Given any other constraint (u,v) satisfying u₀>u and v₀>v,

$$\frac{{u}_{0} - u}{{v}_{0} - v} \geq \frac{{u}_{0} - {u}_{1}}{{v}_{0} - {v}_{1}}$$

or
det(\vec{p}₀,\vec{p}₁)+det(\vec{p},\vec{p}₀)+det(\vec{p}₁,\vec{p})≦0
where \vec{p}=(u,v),\vec{p}₀=(u₀,v₀),\vec{p}₁=(u₁,v₁).
If we write \vec{p}₀>\vec{p} if and only if u₀>u and v₀>v, the pair we seek is the only one that satisfies this determinant inequality for every \vec{p}₀>\vec{p} among the remaining constraints.

### Example 3

### Direct Scoring from Low- and High-Response Distributions

In this example, we analyze the method of scoring unknown wells from known pairs of low and high wells using probability density functions. The model of an interpolant distribution is as described in Examples 1 and 2, above. We assume that the measurements made for each feature come from a continuous probability distribution. The underlying method of scoring calculates the Kolmogorov-Smimov (KS) distance from the unknown test sample (also referred to as a well) to the closest interpolant between the low and high reference samples (wells). The distance between two wells is the maximum of the distances from each feature. A critical feature is the one that achieves this maximum distance.

Given a feature, we let ρ, ρ_{A}, ρ_{B} be the probability density functions of the unknown, low and high distributions for the feature. We shall establish the following facts:

Fact 1. Associated with each feature, there is a (possibly not unique) critical value for the feature, c, independent of the unknown well and only dependent on the low and high wells. This critical value is determined by the property that the likelihood of observations less than c is the same for the low and high wells. This is represented by the equation:

$${\int }_{- ∞}^{c} {ρ}_{A} ( x ) ⅆ x = {\int }_{- ∞}^{c} {ρ}_{B} ( x ) ⅆ x .$$

Fact 2. In case of only one feature, the distance D from the test well to the closest interpolant between the low and high well can be calculated using only the probability density function (“p.d.f”) of the test well, the p.d.f. of the low well and the critical value of the feature. The distance is the absolute difference between the likelihood that an observation in the test well is less than c and the likelihood that an observation in the low well is less than c. This is represented by the equation:

$$D = \vert {\int }_{- ∞}^{c} ρ ( x ) ⅆ x - {\int }_{- ∞}^{c} {ρ}_{A} ( x ) ⅆ x \vert .$$

Fact 3. In case of only one feature, the closest interpolant (the response) to the test well can be calculated from the values of the p.d.f of each well at the critical value of the feature. It is given by the following ratio:

$${α}_{0} = \frac{ρ ( c ) - {ρ}_{A} ( c )}{{ρ}_{B} ( c ) - {ρ}_{A} ( c )} .$$

The calculated response might not be in the interval from 0 to 1 and we may want to impose that constraint on the solution. In that case, the distance is the smallest of the KS distances from the test well to the low and high wells.

Fact 4. In case of more than one feature, the critical features are not unique. The closest interpolant to the test well is a function of two critical features and may not occur at the critical value of either.

Fact 5. The distance D(α) between the test well and the α interpolating distribution is a convex function of a but it may not be differentiable at its minimum.

a) KS Distance Between Two Scalar Distributions.

We assume that the probability distributions in this paper are continuous. The KS distance between the two probability distributions ρ and ρ_{A} is defined by

$$D = {max}_{y} \vert {\int }_{- ∞}^{y} [ ρ ( x ) - {ρ}_{A} ( x ) ] ⅆ x \vert .$$

If we let

$$G ( y ) = {\int }_{- ∞}^{y} [ ρ ( x ) - {ρ}_{A} ( x ) ] ⅆ x$$

and
F(y)=|G(y)|,
then with this notation,

$$D = {max}_{y} F ( y ) .$$

The maximum occurs at an extremal given by

$$0 = \frac{\partial F}{\partial y} = [ ρ ( y ) - {ρ}_{A} ( y ) ] sgn G ( y ) ,$$

where sgn is defined as function that return +1 or −1, depending on the sign of the argument. If ρ and ρ_{A} are not identical, then the maximum is greater than zero and the maximum must occur at values of y where
ρ(y)=ρ_{A}(y).
We call the value of y for which F(y) achieves its maximum, a critical value.
b) KS Distance Between Two Vectors of Distributions.

We define a distance between two vectors of distributions {ρⱼ} and {(ρ_{A})ⱼ} by

$$\begin{matrix}D = {max}_{j} {max}_{y} {F}_{j} ( y ) \\ where \\ {G}_{j} ( y ) = {\int }_{- ∞}^{y} [ {ρ}_{j} ( x ) - {( {ρ}_{A} )}_{j} ( x ) ] ⅆ x\end{matrix}$$

and
Fⱼ(y)=|Gⱼ(y)|.
This distance measures the dissimilarity between the two vectors of distributions; it depends on the largest difference between the corresponding features.

Since the maximum occurs at the maximum of one of the individual features, the maximum must occur at one of the extremal values. We call a feature which achieves the maximum, a critical feature. As before, if the vectors of distributions are not identical, the maximum must occur at a value for y with
ρⱼ(y)=(ρ_{A})ⱼ(y).
The maximum occurs at the critical value of the critical feature.
c) Distance to the Closest Interpolant.

Given a feature, we let ρ, ρ_{A}, ρ_{B} be the probability density functions of the unknown, low and high distributions for the feature. The method of scoring calculates the KS distance from the unknown well to the closest interpolant between the low and high wells. The α interpolating distribution between ρ_{A} and ρ_{B} is defined by
ρ_{α}(x)=αρ_{B}(x)+(1−α)ρ_{A}(x).
The interpolant is only a legal p.d.f. for α in the interval from 0 to 1 (because it takes on negative values outside this interval) but the expression is still valid outside that interval. The distance to the α interpolating distribution is defined by

$$D ( α ) = {max}_{y} \vert {\int }_{- ∞}^{y} [ ρ ( x ) ⅆ x - ( α {ρ}_{B} ( x ) + ( 1 - α ) {ρ}_{A} ( x ) ) ] ⅆ x \vert .$$

If we let

$$G ( α , y ) = {\int }_{- ∞}^{y} [ ρ ( x ) ⅆ x - ( α {ρ}_{B} ( x ) + ( 1 - α ) {ρ}_{A} ( x ) ) ] ⅆ x$$

and
F(α,y)=|G(α,y)|,
then we can write

$$D ( α ) = {max}_{y} F ( α , y ) .$$

The KS distance from the unknown well to the closest interpolant between the low and high wells is then given by

$$D = {min}_{0 \leq α \leq 1} {max}_{y} F ( α , y ) .$$

We want to find saddle points (the minimum in α and maximum in y) of the function
F(α,y)=|G(α,y)|.
The saddle points occur at extrema. One possible extrema is at

$${\int }_{- ∞}^{y} [ ρ ( x ) ⅆ x - ( α {ρ}_{B} ( x ) + ( 1 - α ) {ρ}_{A} ( x ) ] ⅆ x = G ( α , y ) = 0.$$

This can only occur if D=0 and the test well has an identical distribution to one of the interpolants. For now, let us assume that that is not the case and the extremum that we seek is away from the zero of the absolute value function. Then we can examine the zeroes of the two partial derivatives of F:

$$0 = \frac{\partial F}{\partial α} = sgn G {\int }_{- ∞}^{y} ( {ρ}_{B} ( x ) - {ρ}_{A} ( x ) ) ⅆ x$$

$$0 = \frac{\partial F}{\partial y} = sgn G [ ρ ( y ) - {ρ}_{A} ( y ) - α ( {ρ}_{B} ( y ) - {ρ}_{A} ( y ) ) ] .$$

Thus the extremal conditions occur when (α_{c}, c) is a solution to those two equations:

$${\int }_{- ∞}^{c} {ρ}_{A} ( x ) ⅆ x = {\int }_{- ∞}^{c} {ρ}_{B} ( x ) ⅆ x and {α}_{c} = \frac{ρ ( c ) - {ρ}_{A} ( c )}{{ρ}_{B} ( c ) - {ρ}_{A} ( c )} ,$$

assuming ρ_{B}(c)−ρ_{A}(c)≠0. In the case ρ_{B}(c)−ρ_{A}(c)=0, then we must also have ρ(c)−ρ_{A}(c)=0 and all three densities are equal at c. In this case, every α is a solution and the test well is the same distance from all the interpolants and there is no definitive response.

Note that when the extremal conditions are satisfied, the coefficient of a drops out of the equation for D and

$$D = \vert {\int }_{- ∞}^{c} ρ ( x ) ⅆ x - {\int }_{- ∞}^{c} {ρ}_{A} ( x ) ⅆ x \vert = \vert {\int }_{- ∞}^{c} ρ ( x ) ⅆ x - {\int }_{- ∞}^{c} {ρ}_{B} ( x ) ⅆ x \vert .$$

In the special case where the test well has an identical distribution to one of the interpolants, then
ρ(x)−ρ_{A}(x)−α₀(ρ_{B}(x)−ρ_{A}(x))=0
for all x, not just the critical value. Certainly then, the two extremal equations still define a critical value c.

Some extrema occur at minima and some occur at maxima. The distance to the closest interpolant to the unknown distribution is the smallest distance among the extrema which are maxima, the distance to the low distribution and the distance to the high distribution.

A reasonable approach to using a single feature to score a set of unknown wells with a fixed pair of low and high wells is to first calculate the critical values for each feature using only the low and high wells. Given these critical values, the distribution of a given unknown well can be used to determine which critical values correspond to maxima and which correspond to minima. This determination is heavily dependent on the distribution of the unknown well although the possible locations are determined only by the low and high wells.

Note that the function D(α) may not be differentiable at its minimum.

d) Convexity of the Distance.

The function D(α) is a convex function of α. The reason for this is as follows. Let

$$u ( y ) = {\int }_{- ∞}^{y} [ ρ ( x ) ⅆ x - {ρ}_{A} ( x ) ] ⅆ x and v ( y ) = {\int }_{- ∞}^{y} [ {ρ}_{B} ( x ) - {ρ}_{A} ( x ) ] ⅆ x .$$

Consider the set of (D,α) with D≧|u(y)−αv(y)| for every α and y. For fixed y, this is the intersection of two half-spaces in the D−α plane. For all y, the set is the intersection of all these pairs of half-spaces and so is convex. The function D(α) is the boundary curve of this convex set. We have shown that the minimum occurs either when v(y)=0 or at α=0 or at α=1.
e) Multiple Features.

In case there is more than one feature, then the distance is defined to be

$$D = {min}_{0 \leq α \leq 1} {max}_{j} {max}_{y} \vert {\int }_{- ∞}^{y} [ {ρ}_{j} ( x ) ⅆ x - ( {α ( {ρ}_{B} )}_{j} ( x ) + ( 1 - α ) {( {ρ}_{A} )}_{j} ( x ) ) ] ⅆ x \vert$$

where the integer j is designating the feature index. Let

$$D ( α ) = {max}_{j} {max}_{y} \vert {\int }_{- ∞}^{y} [ {ρ}_{j} ( x ) ⅆ x - ( {α ( {ρ}_{B} )}_{j} ( x ) + ( 1 - α ) {( {ρ}_{A} )}_{j} ( x ) ) ] ⅆ x \vert .$$

We shall show that although D(α) is a convex function of α, the minimum is not likely to occur at an extremum of one of the features. In fact, the minimum is generally associated with at least two features.
As before, let

$${u}_{j} ( y ) = {\int }_{- ∞}^{y} [ {ρ}_{j} ( x ) ⅆ x - {( {ρ}_{A} )}_{j} ( x ) ] ⅆ x and$$

$${v}_{j} ( y ) = {\int }_{- ∞}^{y} [ {( {ρ}_{B} )}_{j} ( x ) - {( {ρ}_{A} )}_{j} ( x ) ] ⅆ x .$$

Consider the set of (D,α) with D≧|uⱼ(y)−αvⱼ(y)| for every α and y. This set is the intersection of half-spaces and so is convex.
If we let
Gⱼ(α,y)=uⱼ(y)−αvⱼ(y)
and
Fⱼ(α,y)=|Gⱼ(α,y)|,
then we can write

$${D}_{j} ( α ) = {max}_{y} {F}_{j} ( α , y ) and D ( α ) = {max}_{j} {D}_{j} ( α ) .$$

The continuous convex curve D(α) is comprised of finitely many pieces taken from individual Dⱼ(α). Since it is convex, the minimum must occur at the minimum of one of its pieces or at the intersection of two of the pieces. In the first case, there is one critical feature associated with the minimum and in the second case, there are two. The critical features are the only features necessary to determine the closest interpolant.
f) Calculating the KS Distance to the Closest Interpolant.

We have devised an algorithm for finding a numerical approximation to

$$D = {min}_{0 \leq α \leq 1} D ( α ) .$$

We shall describe the algorithm in terms of a single feature, but it works in similar fashion for any number of features. Corresponding to ρ_{A}, ρ_{B} and ρ, we produce three sets of values {xⱼ}, {yⱼ} and {zⱼ} determined by

$${\int }_{- ∞}^{{x}_{j}} {ρ}_{A} ( x ) ⅆ x = \frac{j}{L} , {\int }_{- ∞}^{{y}_{j}} {ρ}_{A} ( x ) ⅆ x = \frac{j}{M} , {\int }_{- ∞}^{{z}_{j}} {ρ}_{A} ( x ) ⅆ x = \frac{j}{N} .$$

Let sₖ be a member of S={xⱼ}∪{yⱼ}∪{zⱼ}. Let

$${u}_{k} = u ( {s}_{k} ) = {\int }_{- ∞}^{{s}_{k}} [ ρ ( x ) ⅆ x - {ρ}_{A} ( x ) ] ⅆ x , \\ {v}_{k} = v ( {s}_{k} ) = {\int }_{- ∞}^{{s}_{k}} [ {ρ}_{B} ( x ) ⅆ x - {ρ}_{A} ( x ) ] ⅆ x ,$$

Gₖ(α)=uₖ−αvₖ,
and
Fₖ(α)=|Gₖ(α)|.
Method. We approximate D by

$${D}^{⋒} = {min}_{0 \leq α \leq 1} {max}_{k} {F}_{k} ( α ) .$$

i. Using Linear Programming.

The method outlined above involves solving a general problem of this type:

$$D = {min}_{0 \leq α \leq 1} {max}_{k} \vert {u}_{k} - α {v}_{k} \vert$$

where the pairs (uₖ,vₖ) come from finite set.

This program can be stated and solved as a problem in Linear Programming as follows. Note that if the solution D occurs at the value α=α₀, then
D≧↑uₖ−α₀vₖ| for every k.
Thus (D,α₀) is the solution to the LP problem:
min Y(y,α) with Y(y,α)=y subject to
   y−uₖ+vₖα≧0
   y+uₖ−vₖα≧0
   α≦1
   α≧0.
   In particular, if we include the pair (−uₖ,−vₖ) in the set whenever the pair (uₖ,vₖ) is in the set, then we can write the problem as:
min Y(y,α) with Y(y,α)=y subject to
   y−uₖ+vₖα≧0
   α≦1
   α≧0.
   We can use a type of simplex method to solve this problem.

ii. Algorithm

Consider the two-dimensional constraint region with horizontal axis a and vertical axis y. This convex region is bounded by the vertical lines at α=0 and α=1 and lies above all the lines y=−vₖα+uₖ. We will start on the constraint boundary at α=0 and y=max uₖ. Let us assume that the constraint which provides this maximum is called (u₀,v₀). If there is more than one constraint passing through α=0 and y=u₀, we choose the one with the smallest v and hence the largest slope. We follow the constraint (u₀,v₀) until it meets another (u,v) at α=α₀. Then we remove all the constraints (uⱼ,vⱼ) with uⱼ>u because they must meet the constraint (u,v) before α=α₀. Let us assume that this new constraint is called (u₁,v₁). We iterate this procedure with the remaining constraints until either we hit the boundary at α=1 or Y(y,α) starts to increase.

More specifically, we give a method for determining which constraint is the first to meet the constraint (u₀, v₀). To analyze the situation, we solve for the intersection of the constraint (u₁,v₁) and the constraint (u₀,v₀). If these two constraints meet at α=α₀>0 then

$${α}_{0} = {min}_{( u , v )} \frac{{u}_{0} - u}{{v}_{0} - v}$$

and (u₁,v₁) is the (u,v) pair which achieves this minimum.
In particular, since u₀>u₁, we must have v₀>v₁. Given any other constraint (u,v) satisfying u₀>u and v₀>v,

$$\frac{{u}_{0} - u}{{v}_{0} - v} \geq \frac{{u}_{0} - {u}_{1}}{{v}_{0} - {v}_{1}} .$$

Let \vec{p}=(u,v),\vec{p}₀=(u₀,v₀),\vec{p}₁=(u₁,v₁). If we write \vec{p}₀>\vec{p} if and only if u₀>u and v₀>v, the pair we seek is the only one that satisfies this the ratio inequality for every \vec{p}₀>\vec{p} among the remaining constraints.

iii. Accuracy of the Algorithm.

We suppose that

$$F ( {α}_{c} , z ) = {min}_{0 \leq α \leq 1} {max}_{y} F ( α , y ) and$$

$${D}^{⋒} = {F}_{k} ( β ) = {min}_{0 \leq α \leq 1} {max}_{j} {F}_{j} ( α ) .$$

Suppose that sᵢ≦y≦sᵢ₊₁. Let
ρₐ(x)=αρ_{B}(x)+(1−α)ρ_{A}(x).
Then

$$\begin{matrix}F ( α , y ) = \vert {\int }_{- ∞}^{y} [ ρ ( x ) ⅆ x - {ρ}_{a} ( x ) ] ⅆ x \vert \\ = \vert {\int }_{- ∞}^{{s}_{i}} [ ρ ( x ) ⅆ x - {ρ}_{a} ( x ) ] ⅆ x + {\int }_{{s}_{i}}^{y} [ ρ ( x ) ⅆ x - {ρ}_{a} ( x ) ] ⅆ x \vert \leq \\ \vert {\int }_{- ∞}^{{s}_{i}} [ ρ ( x ) ⅆ x - {ρ}_{a} ( x ) ] ⅆ x \vert + \vert {\int }_{{s}_{i}}^{y} [ ρ ( x ) ⅆ x - {ρ}_{a} ( x ) ] ⅆ x \vert \leq \\ \vert {\int }_{- ∞}^{{s}_{i}} [ ρ ( x ) ⅆ x - {ρ}_{a} ( x ) ] ⅆ x \vert + \vert {\int }_{{s}_{i}}^{{s}_{i + 1}} [ ρ ( x ) ⅆ x - {ρ}_{a} ( x ) ] ⅆ x \vert \leq \\ \vert {\int }_{- ∞}^{{s}_{i}} [ ρ ( x ) ⅆ x - {ρ}_{a} ( x ) ] ⅆ x \vert + \frac{1}{n} \leq \\ {max}_{j} \vert {\int }_{- ∞}^{{s}_{j}} [ ρ ( x ) ⅆ x - {ρ}_{a} ( x ) ] ⅆ x \vert + \frac{1}{n}\end{matrix}$$

where
n=min(L,M,N).
Thus,

$$D ( α ) ≡ {max}_{y} F ( α , y ) \leq {max}_{j} \vert {\int }_{- ∞}^{{s}_{j}} [ ρ ( x ) ⅆ x - {ρ}_{a} ( x ) ] ⅆ x \vert + \frac{1}{n} ≡ {D}^{⋒} ( α ) + \frac{1}{n} .$$

In addition,

$${D}^{⋒} ( α ) ≡ {max}_{{s}_{j}} F ( α , {s}_{j} ) \leq {max}_{y} F ( α , y ) \leq D ( α )$$

for all α.
This proves the following Theorem.

Theorem 1. For every α,

$${D}^{⋒} ( α ) \leq D ( α ) \leq {D}^{⋒} ( α ) + \frac{1}{n} .$$

If we substitute β for α in this equation, we get

$$Corollary 1.$$

$${D}^{⋒} \leq D ( β ) \leq {max}_{y} F ( β , y ) \leq {D}^{⋒} + \frac{1}{n} .$$

Additionally, we can show
Corollary 2. For every α,

$$D ( β ) \leq {D}^{⋒} ( α ) + \frac{1}{n} . \\ Proof . \\ {D}^{⋒} ≡ {min}_{0 \leq α \leq 1} {max}_{j} F ( α , {s}_{j} ) \leq {max}_{j} F ( α , {s}_{j} ) ≡ {D}^{⋒} ( α )$$

Clearly,

$$D ≡ F ( {α}_{c} , z ) ≡ {min}_{0 \leq α \leq 1} {max}_{y} F ( α , y ) \leq {max}_{y} F ( β , y ) ≡ D ( β ) , \\ {D}^{⋒} ( {α}_{c} ) ≡ {max}_{{s}_{j}} F ( {α}_{c} , {s}_{j} ) \leq {max}_{y} F ( {α}_{c} , y ) ≡ F ( {α}_{c} , z ) ≡ D and$$

$${D}^{⋒} ≡ {min}_{0 \leq α \leq 1} {max}_{j} F ( α , {s}_{j} ) \leq {max}_{j} F ( {α}_{c} , {s}_{j} ) = {D}^{⋒} ( {α}_{c} ) .$$

Combining all these inequalities gives:

$$Corollary 3.$$

$${D}^{⋒} \leq D \leq {D}^{⋒} + \frac{1}{n} .$$

Finally, we want to bound the error between β and α_{c} as a function of the number of discretization points n. The derivative of D(α) at α_{c} may or may not exist. If we assume that several derivatives along the curve between β and α_{c} exist, we can get some estimates of the error even if the derivatives do not exist α_{c}. The bound on the error depends on how flat the curve is between β and α_{c} and near α_{c}. The flatter the curve, the worse the possible error.

Theorem 2. Let p be a positive even integer (ordinarily, we expect that p=2.) Assume that D(α) hasp continuous derivatives in the open interval from β to α_{c} and that the limit of D′(α) as α approaches α_{c} from β is T. In addition, assume that all derivatives D^{[j]}(α) have zero limit as α approaches α_{c} from β with 1<j<p and that D^{[p]}(α) is larger than some positive value p!M everywhere between β and α_{c}. (We desire the odd derivatives to be zero to ensure the proven convexity of D(α).) Then

$$\vert β - {α}_{c} \vert \leq min ( {( \frac{1}{nM} )}^{\frac{1}{p}} , \frac{1}{nT} ) .$$

Proof. We apply Taylor's Theorem between β and α_{c}. This gives

$$\frac{1}{n} \geq ( D ( β ) - D ( {α}_{c} ) ) = {D}^{′} ( {α}_{c} ) ( β - {α}_{c} ) + \frac{{D}^{[ p ]} ( ξ )}{p !} {( β - {α}_{c} )}^{p} \geq {D}^{′} ( {α}_{c} ) ( β - {α}_{c} ) + {M ( β - {α}_{c} )}^{p} .$$

Because α_{c} is located at the minimum and D(α) is convex, then D′(α_{c}) and β−α_{c} can not have opposite signs. Since each of the two positive terms on the right hand side of this equation cannot be more than 1/n, the result follows easily.
G) Estimating the Response from Sample Distributions

Suppose we are given samples {xᵢⱼ},{yᵢⱼ},{zᵢⱼ} from the three distributions, ρ_{A}, ρ_{B} and ρ. We want to estimate the response α_{c} and the distance D. Fix j and let sⱼ be a member of S={xᵢⱼ}∪{yᵢⱼ}∪{zᵢⱼ}, that is, sⱼ is one of the possible values of the jth statistic. We assume that S is sorted. Define
H(sⱼ)=|{zᵢⱼ:zᵢⱼ<sⱼ}ᵢ₌₁^{N}|/N,
F(sⱼ)=|{yᵢⱼ:yᵢⱼ<sⱼ}ᵢ₌₁^{M}|/M
and
G(sⱼ)=|{xᵢⱼ:xᵢⱼ<sⱼ}ᵢ₌₁^{L}|/L.
As before, using Linear Programming, we calculate

$$\tilde{D} ( α ) = {max}_{j} {max}_{{s}_{j}} \vert \tilde{u} ( {s}_{j} ) - α \tilde{v} ( {s}_{j} ) \vert ,$$

where
ũ(sⱼ)=H(sⱼ)−G(sⱼ)
and
\tilde{v}(sⱼ)=F(sⱼ)−G(sⱼ).

### Example 4

### Multiple Negative Reference Samples

In some assay, it is desirable to control for more than one type of negative control population. For example, if a bioactive compound is applied to a sample in a buffer solution, it may be desirable to measure any response caused by the buffer solution alone, without any of the compound. This results in having two control populations, one not subject to any treatment, and one subject to treatment with the buffer alone. It is desirable to separate the response due solely to the buffer from the total response relative to the untreated negative, so that the effect of the compound alone can be determined. This example provides a method of determining the response due to the perturbation alone.

In order to deal with more than one negative, it is easier to deal with the complimentary response β=(1−α). Let ρ_{N}_{ᵢ} and ρ_{P} be the density functions of the ith negative control and the positive-response populations, respectively, and let ρ_{β} be the density function of a β-interpolant. Under the model of interpolants as linear combinations of the reference responses, the density of the β-interpolant obtained from the ith negative control and positive-response population is
ρ_{β}=βρ_{N}_{ᵢ}+(1−β)ρ_{P}=ρ_{P}+β(ρ_{N}_{ᵢ}−ρ_{P}),
with 0≦β≦1. The density ρ_{β} is the density which is the fraction β of the way from the density ρ_{P} along the vector from ρ_{P} to ρ_{N}_{ᵢ}.

Extending the model of interpolants to the case of multiple negative controls, we have to consider interpolant density functions which are along vectors starting at ρ_{P} but ending at some positive linear combination of the vectors from ρ_{P} each of the density functions ρ_{N}_{ᵢ}. These densities have the form

$${ρ}_{{β}^{->}} = {ρ}_{{β}_{1} , {β}_{2} , … , {β}_{m}} = {ρ}_{p} + {\sum }_{i = 1}^{m} {β}_{i} ( {ρ}_{{N}_{i}} - {ρ}_{p} ) , with$$

$${\sum }_{i = 1}^{m} {β}_{i} \leq 1$$

and βᵢ≧0.
This can be written as

$${ρ}_{{β}^{->}} = {\sum }_{i = 1}^{m} {β}_{i} {ρ}_{{N}_{i}} + {β}_{0} {ρ}_{p} , with$$

$${β}_{0} = 1 - {\sum }_{i = 1}^{m} {β}_{i} .$$

In this case, the response is

$$1 - {β}_{0} = {\sum }_{i = 1}^{m} {β}_{i} .$$

Letting ρ be the test (unknown) distribution, we want to find the closest distribution ρ_{\overline{β}} to ρ. We solve this problem in analogous fashion to the single negative case. We solve the associated Linear Programming problem given by
min Y(y,β₁,β₂, . . . ,βₘ) with Y(y,β₁,β₂, . . . ,βₘ)=y subject to

$$y + {\sum }_{j = 1}^{m} {v}_{kj} {β}_{j} \geq {u}_{k}$$

$$y - {\sum }_{j = 1}^{m} {v}_{kj} {β}_{j} \geq - {u}_{k}$$

$${\sum }_{j = 1}^{m} {β}_{j} \leq 1$$

βⱼ≧0.
The degree of response corresponding to the closest interpolant is

$${\sum }_{j = 1}^{m} {β}_{j} .$$

### Example 5

### Replicate Populations

In some embodiments, multiple samples at a given perturbation level are analyzed. This results in multiple reference fingerprints, multiple test fingerprints, or both. Methods of carrying out the invention in these situations are described below.

a. Multiple Test Fingerprints

In a preferred embodiment, multiple replicates of the test fingerprint are assayed in order to allow for a statistical characterization of an estimate of a response. Each of the multiple test sample fingerprints are scored on the degree of response scale separately, thus giving multiple estimates of the population response. The distribution of the estimates can be analyzed using standard statistical methods to obtain, for example, mean and standard error of the response.

Alternatively, the object feature data obtained from each of the multiple test samples are pooled to create a singe test sample containing data from all the objects from all the samples. The fingerprint of the pooled sample is expected to provide a more accurate estimate of the true test population distribution because of the larger sample size.

b. Multiple Reference Fingerprints

In a preferred embodiment, multiple replicates from one or both reference populations are assayed in order to improve the estimate of the true population distribution(s). The object feature data obtained from each of the replicates samples of a single reference population are pooled to create a singe sample containing the data from all the replicates. The fingerprint of the pooled sample is expected to provide a more accurate estimate of the true population distribution because of the larger sample size.

Alternatively, the fingerprints from each of the reference sample replicates are treated separately. An interpolate scale can be generated from each pair of reference population fingerprints, one sampled from the low-response population and one sampled from high-response reference population. To score a single test fingerprint, the closest interpolant in each of the scales is determined separately, and response scale comprising the closest interpolant is used to score response of the test fingerprint. Alternatively, in order to reduce the computation required, a subset of the possible combinations of low-response and high-response fingerprints are used.

c. Multiple Test and Reference Fingerprints

In a preferred embodiment, the replicates from the reference populations are pooled in order to improve the estimates of the true population distribution. After pooling, the replicate test samples are handled as described above.

### Example 6

### Interpolation Based On Gradual Change Of Cells

This example describes an example of the class of models of intermediate-response interpolants based on the assumption about the underlying biology that each cell responds in a continuous fashion in response to increasing concentration, and that all cells in an intermediate-response population are in the same state. The result is a gradual shift of the feature distributions from the low reference distribution to the high reference distribution.

The model herein is stated in terms of the probability density functions of the population features. In this model, it is assumed that the value of the feature at a fixed percentile changes linearly from the low to the high distribution. This can expressed mathematically as follows.

Let f and g be the density functions of the low and high distributions, respectively, of some feature. Let t be a certain percentile and let x, be the value of the low distribution and x₂ be the value of the high distribution which corresponds to t. That is, the fraction of values of f that are less than x, and the fraction of values of g that are less than x₂ are both t. We write this mathematically as:

$${\int }_{- ∞}^{{x}_{1}} f ( y ) ⅆ y = F ( {x}_{1} ) = t = G ( {x}_{2} ) = {\int }_{- ∞`}^{{x}_{2}} g ( y ) ⅆ y .$$

The assumption of the model is that the cells in the low concentration that have a value x₁ for this feature undergo gradual change to become the cells that have a high value x₂. Thus for an intermediate concentration a percentage α of the way from low to high, we assume that the value associated with the percentile t is given by
x=(1−α)x₁+αx₂.
If we let H_{α} be the cumulative distribution of the intermediate concentration at α, then we can write
H_{α}⁻¹(t)=(1−α)F⁻¹(t)+αG⁻¹(t).

Scoring. Given a test distribution h with cumulative distribution H, we want to see which H_{α} it most closely matches. We have to find

$$D = {min}_{0 \leq α \leq 1} {max}_{x} \vert H ( x ) - {H}_{α} ( x ) \vert .$$

If we substitute t=H_{α}(x), then

$$\begin{matrix}D = {min}_{0 \leq α \leq 1} {max}_{t} \vert H [ {H}_{α}^{- 1} ( t ) ] - t \vert \\ = {min}_{0 \leq α \leq 1} {max}_{t} \vert H [ ( 1 - α ) {F}^{- 1} ( t ) + α {G}^{- 1} ( t ) ] - t \vert .\end{matrix}$$

Finally, if we let t=H(z), then

$$D = {min}_{0 \leq α \leq 1} {max}_{z} \vert H [ ( 1 - α ) {F}^{- 1} H ( z ) + α {G}^{- 1} H ( z ) ] - H ( z ) \vert .$$

This is a mathematical programming problem with a linear objective function and nonlinear constraints. If we let
U(z,α)=H[(1−α)F⁻¹H(z)+αG⁻¹H(z)]−H(z), then

$$D = {min}_{0 \leq α \leq 1} Y$$

subject to
Y≧U(z,α) and
Y≧−U(z,α) for all z.

Discrete Problem. As we have done before, we estimate U(z,α) from the samples of the distributions of low, high and test reagents: {xᵢ}, {yⱼ}, {zₖ}. Let s be any of the values in the collection C={xᵢ}∪{yⱼ}∪{zₖ} and let L, M and N be the number of samples in each set, respectively. We estimate U(s,α) as follows. We take
H(s)=|{zₖ:zₖ<s}|/N,
the fraction of samples of the test distribution which are less than s. Now x=F⁻¹H(s) is the value of the low sample which has the same fraction of low samples less than it as the test distribution has less than s. Similarly y=G⁻¹H(s) is the value of the high sample which has the same fraction of high samples less than it as the test distribution has less than s. Finally,
H[(1−α)F⁻¹H(s)+αG⁻¹H(s)]=|{zₖ:zₖ<(1−α)x+αy}|/N.
Thus we must solve the nonlinear programming problem

$$D = {min}_{0 \leq α \leq 1} Y$$

subject to
Y≧U(s,α) and
Y≧−U(s,α) for all s in C.
The constraints are nonlinear functions
U(s,α)=(|{zₖ:zₖ<(1−α)x+αy}|−|{zₖ:zₖ<s}|)/N
where x and y are fixed values determined by s. Since the value of (1−α)x+αy starts at x and changes linearly to y, U(s,α) is either a monotonically increasing or decreasing function, depending on whether x is less than or more than y. In fact, U(s,α) is piecewise constant function with jumps at the values of the test data.
We solve this problem using approximately the same method that we use for solving a Linear Programming problem.

1. A method of generating a degree of response scale for a population subject to multiple levels of a perturbation, wherein a response is a representation of the multidimensional state of a population, said method comprising the steps of:
a) determining a fingerprint of a first sample of said population subjected to a first level of perturbation, thus obtaining a low-response reference fingerprint that is a representation of the multidimensional state of a low-response population;
b) determining a fingerprint of a second sample of said population subjected to a second level of perturbation greater than said first level of perturbation, thus obtaining a high-response reference fingerprint that is a representation of the multidimensional state of a high-response population;
c) determining a set of interpolated fingerprints from said low-response reference fingerprint and said high-response reference fingerprint, wherein each interpolated fingerprint is a representation of the multidimensional state of a population exhibiting a degree of response intermediate to that of the low-response and high-response populations;
wherein said degree of response scale consists of said set of interpolated fingerprints, said low-response reference fingerprint, and said high-response reference fingerprint, each indexed by a corresponding degree of response.

2. The method of claim 1, wherein the population is a biological population.

3. The method of claim 2, wherein said population is a population of cells.

4. The method of claim 3, wherein determining each of said fingerprints comprises measuring a plurality of features of said cells, wherein at least one of said features is selected from the set of features consisting of length, width, height, perimeter, area, volume, orientation, shape, texture, perimeter, moments, mean, variance, skewness, kurtosis, centroid, color, luminescence, total luminescence, average luminescence, and optical density, wherein each feature is measured independently from either the entire cell or a subregion of said cell.

5. The method of claim 1, wherein said perturbation is chemical, biological, mechanical, thermal, electromagnetic, gravitational, nuclear, or temporal.

6. The method of claim 3, wherein said perturbation is a bioactive compound.

7. The method of claim 1, wherein each interpolated fingerprint of said set of interpolated fingerprints is determined as a linear combination of said low-response reference fingerprint and said high-response reference fingerprint.

8. A method of analyzing the response of a test population subjected to a known level of a test perturbation, relative to a reference perturbation, wherein a response is a representation of the multidimensional state of a population, said method comprising the steps of:
a) determining a degree of response scale by
i) determining a fingerprint of a first sample of said population subjected to a first level of said reference perturbation, thus obtaining a low-response reference fingerprint that is a representation of the multidimensional state of a low-response population;
ii) determining a fingerprint of a second sample of said population subjected to a second level of said reference perturbation greater than said first level of perturbation, thus obtaining a high-response reference fingerprint that is a representation of the multidimensional state of a high-response population;
iii) determining a set of interpolated fingerprints from said low-response reference fingerprint and said high-response reference fingerprint, wherein each interpolated fingerprint is a representation of the multidimensional state of a population exhibiting a degree of response intermediate to that of the low-response and high-response populations;
wherein said degree of response scale consists of said set of interpolated fingerprints, said low-response reference fingerprint, and said high-response reference fingerprint, each indexed by a corresponding degree of response;
b) determining a fingerprint of said test population subjected to said known level of a test perturbation, thus obtaining a test fingerprint that is a representation of the multidimensional state of said test population; and
c) determining a degree of response of said test population by determining from among said degree of response scale a fingerprint most similar to said test fingerprint and identifying a degree of response corresponding to said most similar fingerprint.

9. The method of claim 8, wherein said reference and test populations are biological populations.

10. The method of claim 9, wherein said reference and test populations are populations of cells.

11. The method of claim 10, wherein determining each of said fingerprints comprises measuring a plurality of features of said cells, wherein at least one of said features is selected from the set of features consisting of length, width, height, perimeter, area, volume, orientation, shape, texture, perimeter, moments, mean, variance, skewness, kurtosis, centroid, color, luminescence, total luminescence, average luminescence, and optical density, wherein each feature is measured independently from either the entire cell or a subregion of said cell.

12. The method of claim 8, wherein said perturbation is chemical, biological, mechanical, thermal, electromagnetic, gravitational, nuclear, or temporal.

13. The method of claim 9, wherein said perturbation is a bioactive compound.

14. The method of claim 8, wherein said test perturbation and said reference perturbation are the same.

15. The method of claim 8, wherein said test perturbation and said reference perturbation are different.

16. The method of claim 8, wherein each interpolated fingerprint of said set of interpolated fingerprints is determined as a linear combination of said low-response reference fingerprint and said high-response reference fingerprint.

17. A method of generating a dose-response relationship for a population subjected to a perturbation, wherein a response is a representation of the multidimensional state of a population, said method comprising the steps of:
a) determining a degree of response scale by
i) determining a fingerprint of a first sample of said population subjected to a first level of perturbation, thus obtaining a low-response reference fingerprint that is a representation of the multidimensional state of a low-response population;
ii) determining a fingerprint of a second sample of said population subjected to a second level of perturbation greater than said first level of perturbation, thus obtaining a high-response reference fingerprint that is a representation of the multidimensional state of a high-response population; and
iii) determining a set of interpolated fingerprints from said low-response reference fingerprint and said high-response reference fingerprint, wherein each interpolated fingerprint is a representation of the multidimensional state of a population exhibiting a degree of response intermediate to that of the low-response and high-response populations;
wherein said degree of response scale consists of said set of interpolated fingerprints, said low-response reference fingerprint, said high-response reference fingerprint, each indexed by a corresponding degree of response;
b) determining a plurality of fingerprints of a plurality of test samples of said population, each subjected a different known level of said test perturbation, thus obtaining a test fingerprint corresponding to each of a plurality of levels of perturbation;
c) determining a degree of response for each test fingerprint by determining from among said degree of response scale a fingerprint most similar to said test fingerprint and identifying a degree of response corresponding to said most similar fingerprint;
wherein said dose-response relationship is represented by said degree of response obtained for each of said plurality of levels of perturbation.

18. A method of generating a dose-response curve for a population subjected to a perturbation, wherein said method comprises generating a dose-response relationship for said population according to the method of claim 15, and fitting a curve to the results obtained.
